(19) United States
(12) Patent Application Publication
(10) Pub. No.: US 2023/0165672 A1
(43) Pub. Date: Jun. 1, 2023

(54) ADJUSTABLE INTERATRIAL DEVICES, AND ASSOCIATED SYSTEMS AND METHODS

(71) Applicant: Shifamed Holdings, LLC, Campbell, CA (US)

(72) Inventors: Brian Fahey, Menlo Park, CA (US); Scott Robertson, Portland, OR (US); Miles Alexander, Fremont, CA (US); Anthony Pantages, San Jose, CA (US); Tom Saul, Moss Beach, CA (US); William Jason Fox, San Mateo, CA (US)

(21) Appl. No.: 17/918,273

(22) PCT Filed: Apr. 16, 2021
(86) PCT No.: PCT/US2021/027747

§ 371 Date: Oct. 11, 2022

Related U.S. Application Data
(60) Provisional application No. 63/010,841, filed on Apr. 16, 2020.

(51) Int. Cl. A61F 2/06

(52) U.S. Cl. CPC A61F 2/06; A61F 2002/068; A61F 2250/001; A61F 2210/0014

(57) ABSTRACT

The present technology relates to adjustable interatrial shunts that are configured to shunt blood between the left atrium and the right atrium. In particular, the adjustable interatrial shunts can include a flow control element moveable through a plurality of discrete position, with each discrete position being associated with a particular shunt geometry, and with each particular shunt geometry being associated with a different fluid resistance and/or relative drainage resistance through the shunt for a given pressure differential between the left atrium and the right atrium. The flow control element can be selectively moveable between the plurality of discrete positions by operation of an actuation assembly. In some embodiments, the adjustable interatrial shunts include a ratchet mechanism that controls the movement of flow control element through the plurality of discrete positions, and can hold or lock the shunt in a desired position or configuration.

### CROSS-REFERENCE TO RELATED APPLICATION(S)

This application claims the benefit of U.S. Provisional Patent Application No. 63/010,841, filed Apr. 16, 2020, and incorporated herein by reference in its entirety.

### TECHNICAL FIELD

The present technology generally relates to implantable medical devices and, in particular, to implantable interatrial systems and associated methods for selectively controlling blood flow between the right atrium and the left atrium of a heart.

### BACKGROUND

Heart failure is a medical condition associated with the inability of the heart to effectively pump blood to the body. Heart failure affects millions of people worldwide, and may arise from multiple root causes, but is generally associated with myocardial stiffening, myocardial shape remodeling, and/or abnormal cardiovascular dynamics. Chronic heart failure is a progressive disease that worsens considerably over time. Initially, the body's autonomic nervous system adapts to heart failure by altering the sympathetic and parasympathetic balance. While these adaptations are helpful in the short-term, over a longer period of time they may serve to make the disease worse.

Heart failure (HF) is a medical term that includes both heart failure with reduced ejection fraction (HFrEF) and heart failure with preserved ejection fraction (HFpEF). The prognosis with both HFpEF and HFrEF is poor; one-year mortality is 26% and 22%, respectively, according to one epidemiology study. In spite of the high prevalence of HFpEF, there remain limited options for HFpEF patients. Pharmacological therapies have been shown to impact mortality in HFrEF patients, but there are no similarly-effective evidence-based pharmacotherapies for treating HFpEF patients. Current practice is to manage and support patients while their health continues to decline.

A common symptom among heart failure patients is elevated left atrial pressure. In the past, clinicians have treated patients with elevated left atrial pressure by creating a shunt between the left and right atria using a blade or balloon septostomy. The shunt decompresses the left atrium (LA) by relieving pressure to the right atrium (RA) and systemic veins. Over time, however, the shunt typically will close or reduce in size. More recently, percutaneous interatrial shunt devices have been developed which have been shown to effectively reduce left atrial pressure. However, these percutaneous devices have an annular passage with a fixed diameter which fails to account for a patient's changing physiology and condition. For this reason, existing percutaneous shunt devices may have a diminishing clinical effect after a period of time. Many existing percutaneous shunt devices typically are also only available in a single size that may work well for one patient but not another. Also, sometimes the amount of shunting created during the initial procedure is later determined to be less than optimal months later. Accordingly, there is a need for improved devices, systems, and methods for treating heart failure patients, particularly those with elevated left atrial pressure.

### BRIEF DESCRIPTION OF THE DRAWINGS

FIG. 1 is a schematic illustration of an interatrial device implanted in a heart and configured in accordance with select embodiments of the present technology.

FIGS. 2A-2B illustrate an interatrial shunting system configured in accordance with an embodiment of the present technology.

FIGS. 3A-3D illustrate an interatrial shunting system configured in accordance with an embodiment of the present technology.

FIGS. 4A-4C illustrate an actuation assembly of the interatrial shunting system illustrated in FIGS. 3A-3D, and configured in accordance with an embodiment of the present technology.

FIGS. 5A-5C illustrate an actuation assembly configured in accordance with an embodiment of the present technology.

FIGS. 6A-6D illustrate an actuation assembly configured in accordance with an embodiment of the present technology.

FIGS. 7A and 7B illustrate an actuation assembly configured in accordance with an embodiment of the present technology.

### DETAILED DESCRIPTION

The present technology is generally directed to implantable systems and devices for facilitating the flow of fluid between a first body region and a second body region. In embodiments, the devices are selectively adjustable to control the amount of fluid flowing between the first body region and the second body region. The devices generally include a drainage and/or shunting element having a lumen extending therethrough for draining or otherwise shunting fluid between the first and second body regions. Some embodiments include an actuation assembly that can drive movement of a flow control element to change the flow resistance through the lumen or another characteristic of the lumen, thereby increasing or decreasing the relative drainage or flow rate of fluid between the first body region and the second body region.

In particular, some embodiments of the present technology provide adjustable devices that are selectively titratable to provide various levels of therapy. For example, the devices can be adjusted through a number of discrete positions or configurations, with each position or configuration providing a different flow resistance and/or drainage rate relative to the other positions or configurations. Accordingly, the devices can be incrementally adjusted through the positions or configurations until the desired flow resistance and/or drainage rate is achieved. Once the desired flow resistance and/or drainage rate is achieved, the devices are configured to maintain the set position or configuration until further input. In some embodiments, various components of the devices operate as a ratchet and/or similar to a hemostat mechanism, which enables the incremental adjustments of the devices between the plurality of positions or configurations, and can hold or lock the device in the desired position or configuration.

In some embodiments, the present technology provides adjustable interatrial shunts that are configured to shunt blood from the left atrium (LA) to the right atrium (RA). The adjustable interatrial shunts can include a shunting element having a lumen extending therethrough and configured to fluidly connect the LA and the RA. The adjustable interatrial shunts can further include a flow control element operably coupled to the shunt. The flow control element can be moveable through a plurality of discrete positions, with each discrete position being associated with a particular shunt geometry, and with each particular shunt geometry being associated with a different relative drainage resistance through the lumen for a given pressure differential between the LA and the RA. The flow control element can be selectively moveable between the plurality of discrete positions by operation of an actuation assembly. In some embodiments, the adjustable interatrial shunts include a ratchet mechanism that controls the movement of flow control element through the plurality of discrete positions and can hold or lock the shunt in a desired position or configuration.

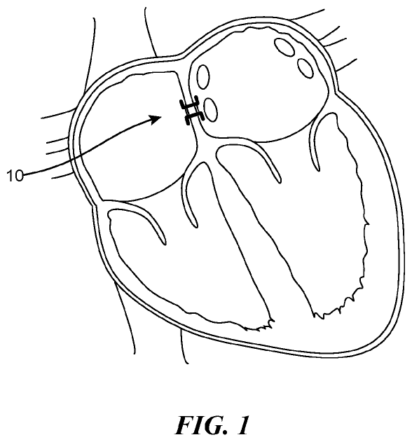

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to FIGS. 1-7B.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

As used herein, the terms “interatrial device,” “interatrial shunt device,” “IAD,” “IASD,” “interatrial shunt,” and “shunt” are used interchangeably to refer to a device that, in at least one configuration, includes a shunting element that provides a blood flow between a first region (e.g., a LA of a heart) and a second region (e.g., a RA or coronary sinus of the heart) of a patient. Although described in terms of a shunt between the atria, namely the left and right atria, one will appreciate that the technology may be applied equally to devices positioned between other chambers and passages of the heart, or between other parts of the cardiovascular system. For example, any of the shunts described herein, including those referred to as “interatrial,” may be nevertheless used and/or modified to shunt between the LA and the coronary sinus, or between the right pulmonary vein and the superior vena cava. Moreover, while the disclosure herein primarily describes shunting blood from the LA to the RA, the present technology can be readily adapted to shunt blood from the RA to the LA to treat certain conditions, such as pulmonary hypertension. For example, mirror images of embodiments, or in some cases identical embodiments, used to shunt blood from the LA to the RA can be used to shunt blood from the RA to the LA in certain patients. Additionally, the technology described herein can be used to shunt fluids other than blood (e.g., cerebrospinal fluid, aqueous humor, etc.) between other body regions.

As used herein, the term “geometry” can include both the size and/or the shape of an element. Accordingly, when the present disclosure describes a change in geometry, it can refer to a change in the size of an element (e.g., moving from a smaller circle to a larger circle), a change in the shape of an element (e.g., moving from a circle to an oval), and/or a change in the shape and size of an element (e.g., moving from a smaller circle to a larger oval).

The headings provided herein are for convenience only and do not interpret the scope or meaning of the claimed present technology.

### A. Interatrial Shunts for Treatment of Heart Failure

Heart failure can be classified into one of at least two categories based upon the ejection fraction a patient experiences: (1) HFpEF, historically referred to as diastolic heart failure or (2) HFrEF, historically referred to as systolic heart failure. One definition of HFrEF is a left ventricular ejection fraction lower than 35%-40%. Though related, the underlying pathophysiology and the treatment regimens for each heart failure classification may vary considerably. For example, while there are established pharmaceutical therapies that can help treat the symptoms of HFrEF, and at times slow or reverse the progression of the disease, there are limited available pharmaceutical therapies for HFpEF with only questionable efficacy.

In heart failure patients, abnormal function in the left ventricle (LV) leads to pressure build-up in the LA. This leads directly to higher pressures in the pulmonary venous system, which feeds the LA. Elevated pulmonary venous pressures push fluid out of capillaries and into the lungs. This fluid build-up leads to pulmonary congestion and many of the symptoms of heart failure, including shortness of breath and signs of exertion with even mild physical activity. Risk factors for HF include renal dysfunction, hypertension, hyperlipidemia, diabetes, smoking, obesity, old age, and obstructive sleep apnea. HF patients can have increased stiffness of the LV which causes a decrease in left ventricular relaxation during diastole resulting in increased pressure and inadequate filling of the ventricle. HF patients may also have an increased risk for atrial fibrillation and pulmonary hypertension, and typically have other comorbidities that can complicate treatment options.

Interatrial shunts have recently been proposed as a way to reduce elevated left atrial pressure, and this emerging class of cardiovascular therapeutic interventions has been demonstrated to have significant clinical promise. FIG. 1, for example, shows the conventional placement of a shunt in the septal wall between the LA and RA. Most conventional interatrial shunts (e.g., shunt 10) involve creating a hole or inserting a valve with a lumen into the atrial septal wall, thereby creating a fluid communication pathway between the LA and the RA. As such, elevated left atrial pressure may be partially relieved by unloading the LA into the RA. In early clinical trials, this approach has been shown to improve symptoms of heart failure.

One challenge with many conventional interatrial shunts is determining the most appropriate size and shape of the shunt lumen. A lumen that is too small may not adequately unload the LA and relieve symptoms; a lumen that is too large may overload the RA and right-heart more generally, creating new problems for the patient. Moreover, the relationship between pressure reduction and clinical outcomes and the degree of pressure reduction required for optimized outcomes is still not fully understood, in part because the pathophysiology for HFpEF (and to a lesser extent, HFrEF) is not completely understood. As such, clinicians are forced to take a best guess at selecting the appropriately sized shunt (based on limited clinical evidence) and generally cannot adjust the sizing over time. Worse, clinicians must select the size of the shunt based on general factors (e.g., the size of the patient's anatomical structures, the patient's hemodynamic measurements taken at one snapshot in time, etc.) and/or the design of available devices rather than the individual patient's health and anticipated response. With such traditional devices, the clinician does not have the ability to adjust or titrate the therapy once the device is implanted, for example, in response to changing patient conditions such as progression of disease. By contrast, interatrial shunting systems configured in accordance with embodiments of the present technology allow a clinician to select the size—perioperatively or post-implant—based on the patient.

### B. Interatrial Shunting Systems

In some embodiments, the present technology provides adjustable interatrial shunts that are configured to shunt blood from the LA to the RA. The adjustable interatrial shunts can include a shunting element having a lumen extending therethrough and configured to fluidly connect the LA and the RA. The adjustable interatrial shunts can further include a flow control element operably coupled to the shunt. The flow control element can be moveable through a plurality of discrete positions, with each discrete position being associated with a particular shunt geometry, and with each particular shunt geometry being associated with a different relative drainage resistance through the lumen for a given pressure differential between the LA and the RA. The flow control element can be selectively moveable between the plurality of discrete positions by operation of an actuation assembly. In some embodiments, the adjustable interatrial shunts include a ratchet mechanism and/or a mechanism similar to a hemostat that controls the movement of flow control element through the plurality of discrete positions, and can hold or lock the shunt in a desired position or configuration.

In some embodiments, the flow control element is configured to change a flow resistance through the shunting element to alter the flow of fluid through the lumen. For example, the flow control element can be configured to change a size, shape, or other dimension of a portion (e.g., an orifice such as an outflow or inflow port) of the lumen. In some embodiments, the flow control element can selectively change a size and/or shape of an orifice to alter the flow through the lumen. For example, the flow control element can be configured to selectively increase a diameter of the orifice and/or selectively decrease a diameter of the orifice (or another portion of the lumen) in response to an input. Throughout the present disclosure, reference to adjusting a diameter (e.g., increasing a diameter, decreasing a diameter, etc.) can refer to adjusting a hydraulic diameter of the lumen, adjusting a diameter at a particular location of the lumen, and/or adjusting a diameter along a length (e.g., a full length) of the lumen. In other embodiments, the flow control element is configured to otherwise affect a shape of the lumen. Accordingly, the flow control element can be coupled to a shunting element and/or can be included within the shunting element. For example, in some embodiments the flow control element is part of the shunting element and at least partially defines the orifice. In other embodiments, the flow control element is spaced apart from but is operably coupled to the shunting element.

In some embodiments, the systems described herein can include one or more actuation elements coupled to the flow control element. The flow control element can at least partially define a lumen orifice through which fluid traveling through the interatrial device must pass. Movement of the actuation element(s) may generate a change in a geometry of the flow control element, and thus a change in geometry of the fluid path. The change in geometry can be a restriction (e.g., contraction), an opening (e.g., expansion), or another configuration change.

In some embodiments, the actuation element can include a shape memory material (e.g., a shape memory alloy, or a shape memory polymer). Movement of an actuation element can be generated through externally applied stress and/or the use of a shape memory effect (e.g., as driven by a change in temperature). The shape memory effect enables deformations that have altered an element from its shape-set geometric configuration to be largely or entirely reversed during operation of the actuation element. For example, sufficient heating can produce at least a temporary change in material state (e.g., a phase change) in the actuator material, inducing a temporary elevated internal stress that promotes a shape change toward the original shape-set geometric configuration. In an example, the geometric change that accompanies this change in material state may reverse deformations that have been made to the material following manufacturing. For a shape memory alloy, the change in state can be from a martensitic phase (alternatively, R-phase) at the lower temperature to an austenitic phase (alternatively, R-phase) at the higher temperature. For a shape memory polymer, the change in state can be via a glass transition temperature or a melting temperature. The change in material state can recover deformation(s) of the material—for example, deformation with respect to its original (e.g., manufactured) geometric configuration—without any externally applied stress to the actuator element. That is, a deformation that is present in the material at a first temperature (e.g., body temperature) can be recovered and/or altered by raising the material to a second (e.g., higher) temperature. In some embodiments, upon cooling (and re-changing material state, e.g., back to a martensitic phase), the actuator element may approximately retain its geometric configuration (e.g., it may remain in the configuration that results from the application of heat). In some embodiments, upon cooling the actuator element may approximately retain its geometric configuration to within 30% of the heated, phase transition configuration. However, when the material has returned to a relatively cooler temperature (e.g., cools following the cessation of heat application), it may require a relatively lower force or stress to thermoelastically deform it compared to the material at a sufficiently heated temperature, and as such any subsequently applied external stress can cause the actuator element to once again deform away from the original geometric configuration.

The shape memory actuation element can be processed such that a transition temperature at which the change in state occurs (e.g., the austenite start temperature, the austenite final temperature, etc.) is above a threshold temperature (e.g., body temperature). For example, the transition temperature can be set to be about 45 deg. C., about 50 deg. C., about 55 deg. C., about 60 deg. C., or another higher or lower temperature. In some embodiments, the actuator material is heated from body temperature to a temperature above the austenite start temperature (or alternatively above the R-phase start temperature) such that an upper plateau stress (e.g., “UPS_body temperature”) of the material in a first state (e.g., thermoelastic martensitic phase, or thermoelastic R-phase at body temperature) is lower than an upper plateau stress (e.g., “UPS_actuated temperature”) of the material in a heated state (e.g., superelastic state), which achieves partial or full free recovery. For example, the actuator material can be heated such that UPS_actuated temperature>UPS_body temperature. In some embodiments, the actuator material is heated from body temperature to a temperature above the austenite start temperature (or alternatively above the R-phase start temperature) such that an upper plateau stress of the material in a first state (e.g., thermoelastic martensite or thermoelastic R-phase at body temperature”) is lower than a lower plateau stress (e.g., “LPS”) of the material in a heated state (e.g., superelastic state), which achieves partial or full free recovery. For example, the actuator material can be aged such that LPS_activated temperature>UPS_body temperature. In some embodiments, the actuator material is heated from body temperature to a temperature above the austenite start temperature (or alternatively above the R-phase start temperature) such that an upper plateau stress of the material in a first state (e.g., thermoelastic martensite or thermoelastic R-phase) is higher than a lower plateau stress of the material in a heated state, which achieves partial free recovery. For example, the actuator material can be aged such that LPS_activated temperature<UPS_body temperature.

As one of skill in the art will appreciate from the disclosure herein, various components of the interatrial shunting systems described above can be omitted without deviating from the scope of the present technology. Likewise, additional components not explicitly described above may be added to the interatrial shunting systems without deviating from the scope of the present technology. Accordingly, the systems described herein are not limited to those configurations expressly identified, but rather encompasses variations and alterations of the described systems.

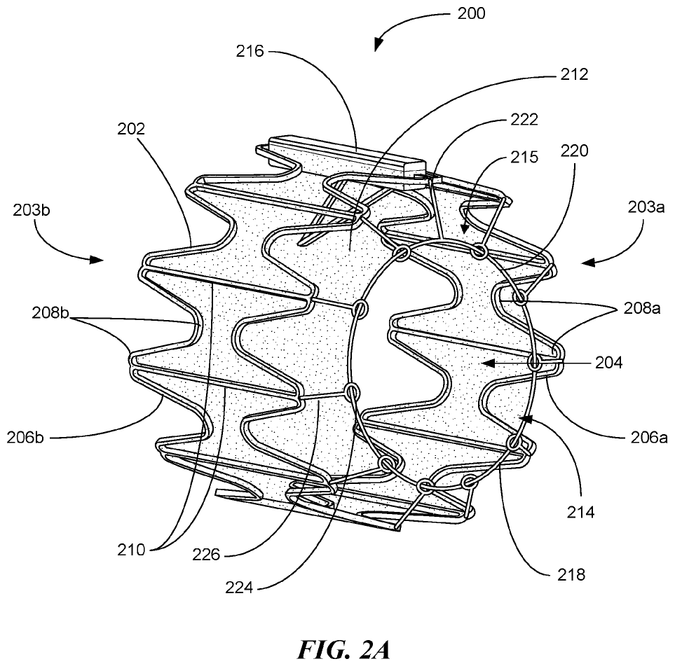

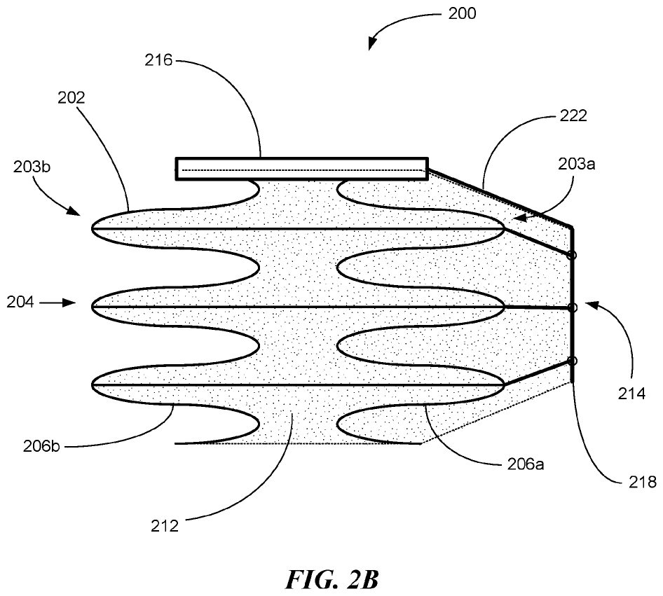

FIGS. 2A and 2B illustrate an interatrial shunting system 200 configured in accordance with an embodiment of the present technology. More specifically, FIG. 2A is a perspective view of the system 200 and FIG. 2B is a side view of the system 200. Referring to FIGS. 2A and 2B together, the system 200 includes a shunting element 202 defining a lumen 204 therethrough. The shunting element 202 can include a first end portion 203a configured to be positioned in or near the LA (not shown) and a second end portion 203b configured to be positioned in or near the RA (not shown). Accordingly, when implanted in the septal wall (not shown) of a patient, the system 200 fluidly connects the LA and the RA via the lumen 204. In some embodiments, the system 200 serves as a sub-system that interfaces with additional structures (not shown), for example, anchoring and/or frame components, to form an interatrial shunting system configured in accordance with an embodiment of the present technology.

The shunting element 202 can be a frame structure including a first annular element 206a at the first end portion 203a and a second annular element 206b at the second end portion 203b. The first and second annular elements 206a-b can each extend circumferentially around the lumen 204. In the illustrated embodiment, the first and second annular elements 206a-b each have a serpentine shape with a plurality of respective apices 208a-b. The apices 208a-b can be curved or rounded. In other embodiments, the apices 208a-b can be pointed or sharp such that the first and second annular elements 206a-b have a zig-zag shape. Optionally, the first and second annular elements 206a-b can have different and/or irregular patterns of apices 208a-b, or can be entirely devoid of apices 208a-b. The first and second annular elements 206a-b can be coupled to each other by one or more struts 210 extending longitudinally along the shunting element 202. The struts 210 can be positioned between the respective apices 208a-b of the first and second annular elements 206a-b. Other suitable stent like configurations may also be used to form the shunting element 202.

The system 200 further includes a membrane 212 operably coupled (e.g., affixed, attached, or otherwise connected) to the shunting element 202. In some embodiments, the membrane 212 is flexible and can be made of a material that is impermeable to or otherwise resists blood flow therethrough. In some embodiments, for example, membrane 212 can be made of a thin, elastic material such as a polymer. For example, the membrane 212 can be made of polytetrafluoroethylene (PTFE), expanded PTFE (ePTFE), silicone, nylon, polyethylene terephthalate (PET), polyether block amide (pebax), polyurethane, blends or combinations of these materials, or other suitable materials.

The membrane 212 can cover or otherwise interface with at least a portion of the shunting element 202, such as the exterior surface of the shunting element 202 between the first end portion 203a and the second end portion 203b. The membrane 212 can extend circumferentially around the shunting element 202 to at least partially surround and enclose the lumen 204. For example, in the illustrated embodiment, the membrane 212 extends between the first and second annular elements 206a-b and over the struts 210. The membrane 212 can couple the first and second annular elements 206a-b to each other, in combination with or as an alternative to the struts 210. The membrane 212 can extend past the first end portion 203a and/or the first annular element 206a (e.g., as best seen in FIG. 2B) so that a portion of the membrane 212 is positioned over and partially covers the lumen 204. In some embodiments, the membrane 212 does not extend past the second end portion 203b and/or the second annular element 206b.

The membrane 212 includes an aperture 214 formed therein. When the membrane 212 is coupled to the shunting element 202, the aperture 214 can be at least generally aligned with or otherwise overlap the lumen 204 to permit blood flow therethrough. In some embodiments, the aperture 214 is positioned at or near the first end portion 203a of the shunting element 202. In other embodiments, the aperture 214 can be positioned at or near the second end portion 203b. Additionally, although FIG. 2A illustrates the aperture 214 as having an elliptical shape, in other embodiments the aperture 214 can have a different shape, such as a circular, square, rectangular, polygonal, or curvilinear shape.

The geometry (e.g., size and/or shape) of the aperture 214 can be varied by deforming (e.g., stretching and/or compressing) or otherwise moving the portions of the membrane 212 surrounding the aperture 214. The change in geometry of the aperture 214 can affect the flow resistance and/or the amount of blood flow through the lumen 204. In some embodiments, depending on the size of the aperture 214 relative to the size of the lumen 204, blood flow through the lumen 204 can be partially or completely obstructed by the membrane 212. Accordingly, an increase in the size (e.g., a diameter, an area) of the aperture 214 can increase the amount of blood flow through the lumen 204 (e.g., by decreasing the flow resistance through the lumen 204), while a decrease in the size of the aperture 214 can decrease the amount of blood flow (e.g., by increasing the flow resistance through the lumen 204).

The system 200 can include an actuation assembly 216 operably coupled to the aperture 214 to selectively adjust the size thereof. In some embodiments, the actuation assembly 216 is coupled to a flow control element 215 that can adjust the geometry of the aperture 214. In the illustrated embodiment, the flow control element 215 includes a string element 218 (e.g., a cord, thread, fiber, wire, tether, ligature, or other flexible elongated element) around the aperture 214 for controlling the size thereof. For example, the string element 218 can include a loop portion 220 surrounding the aperture 214 and a connecting portion 222 coupling the loop portion 220 to the actuation assembly 216. In some embodiments, the loop portion 220 and the connecting portion 222 are different portions of one contiguous elongated element (e.g., arranged similarly to a lasso or snare) that attain their relative shapes (e.g., an elliptical, loop-like shape) as a consequence of how they are connected to the system 200. In other embodiment, the loop portion 220 and the connecting portion 222 can be separate elements that are directly or indirectly coupled to each other.

One or more portions of the string element 218 (e.g., the loop portion 220) can be coupled to the portion of the membrane 212 near the aperture 214. In the illustrated embodiment, the string element 218 (e.g., loop portion 220) passes through a plurality of openings or holes 224 (e.g., eyelets) located near the peripheral portion of the aperture 214. The openings 224 can be coupled to the shunting element 202 (e.g., to the first end portion 203a and/or first annular element 206a) via a plurality of flexible ribs 226 (e.g., sutures, strings, threads, metallic structures, polymeric structures, etc.). In other embodiments, the openings 224 are formed in or coupled directly to the membrane 212 such that the ribs 226 are omitted.

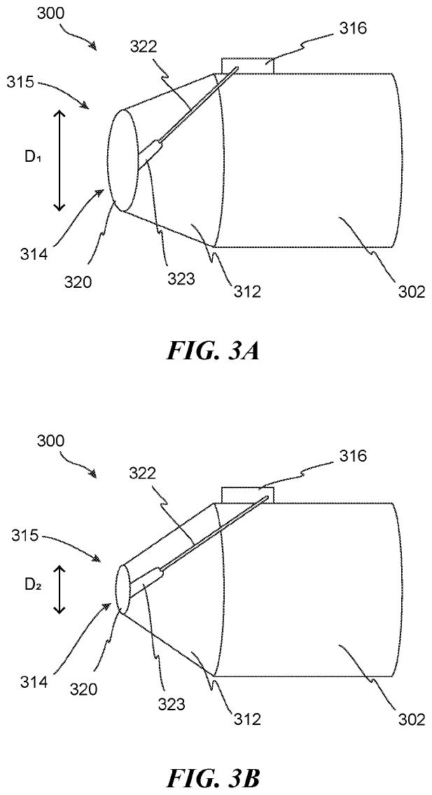

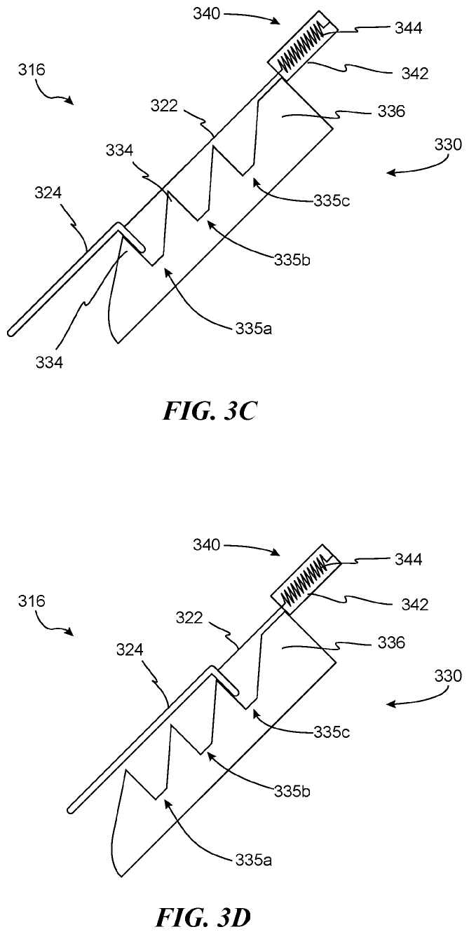

In some embodiments, the string element 218 has a lasso- or noose-like configuration in which the loop portion 220 can be tightened to a smaller size or loosened to a larger size by making an adjustment to (e.g., translating, rotating, applying or releasing tension, etc.) the connecting portion 222. In some embodiments, a motion caused by the adjustment of connecting portion 222 creates an induced motion in loop portion 220 (e.g., a motion that results in the loop portion 220 shifting to a larger or a smaller size). Due to the coupling between the string element 218 and the membrane 212, the size of the aperture 214 (e.g., a diameter, an area) can change along with the size of the loop portion 220 such that the size of the aperture 214 increases as the size of the loop portion 220 increases, and decreases as the size of the loop portion 220 decreases. For example, as the size of the loop portion 220 decreases, the portions of the membrane 212 surrounding the aperture 214 can be cinched, stretched, or otherwise drawn together by the loop portion 220 so that the size of the aperture 214 decreases. Conversely, as the size of the loop portion 220 increases, the portions of the membrane 212 surrounding the aperture can be released, loosened, stretched, or otherwise allowed to move apart so that the size of the aperture 214 increases. As described in greater detail with reference to FIGS. 3A-3D, the actuation assembly 216 can adjust the size of the loop portion 220, and thus the size of the aperture 214, by controlling the amount of force (e.g., tension) applied to the loop portion 220 via the connecting portion 222. For example, in some embodiments, the actuation assembly 216 increases the size of the loop portion 220 and aperture 214 by increasing the amount of force applied to the connecting portion 222, and decreases the size of the loop portion 220 and aperture 214 by decreasing the amount of applied force.

In other embodiments, the system 200 can implement different mechanisms for mechanically and/or operably coupling the actuation assembly 216, the loop portion 220, and the connecting portion 222. For example, there can be an inverse relationship between these components, e.g., the actuation assembly 216 can increase the size of the loop portion 220 and aperture 214 by increasing the amount of force applied to the connecting portion 222, and can decrease the size of the loop portion 220 and aperture 214 by decreasing the amount of applied force. In some embodiments, changes in the size of the loop portion 220 and aperture 214 are created via the actuation assembly 216 translating, rotating, or otherwise manipulating the connecting portion 222 in a way that does not substantially increase or decrease the amount of force applied to the connecting portion 222. In other embodiments, the adjustment to the connecting portion 222 made by the actuation assembly 216 can result in an alteration of the shape of (rather than the size of) loop portion 220 and aperture 214.

In some embodiments, the connecting portion 222 can be surrounded by or otherwise interface with a relatively stiff stabilization element (e.g., a conduit such as a plastic or metallic hypotube—not shown in FIGS. 2A-2B, see, e.g., FIGS. 3A-3B) that can facilitate the transfer of forces from the actuation assembly 216. The stabilization element can be flexible or hinged such that it can move with one or more degrees of freedom with respect to the actuation assembly 216 and/or the aperture 214. In such embodiments, a change in the position of or the tension of connecting portion 222 induced by actuation element 216 may be translated to loop portion 220 in a more consistent manner. For example, the stabilization element may help minimize the shape changes induced in aperture 214 and bias any changes produced in the loop portion 220 by the connecting portion 222 to be manifested predominantly via a change in size (e.g., moving from a larger diameter oval with similar length major and minor axes to a similarly-shaped but smaller diameter oval) as opposed to a change in shape (e.g., moving from a larger diameter oval with similar length major and minor axes to a differently shaped geometry, for instance an oval with substantially different length major and minor axes).

The actuation assembly 216 can be configured in a number of different ways. In some embodiments, for example, the actuation assembly 216 can include one or more shape memory elements configured to change geometry (e.g., transform between a first configuration and a second configuration) in response to a stimulus (e.g., heat or mechanical loading) as is known to those of skill in the art. It will be appreciated that many different types of shape changes can be produced via a shape memory effect. Accordingly, although certain embodiments herein are described in terms of transforming between a shortened configuration and a lengthened configuration, this is not intended to be limiting, and one of skill in the art will appreciate that the present technology can incorporate other types of shape changes produced via a shape memory effect. In some embodiments, the actuation assembly 216 can include one or more motors, such as electromagnetic motors, implanted battery and mechanical motors, MEMS motors, micro brushless DC motors, piezoelectric based motors, solenoids, and other motors. Furthermore, as described in greater detail below with references to FIGS. 3A-3D, the actuation assembly 216 may incorporate a ratchet mechanism and/or mechanisms similar to a hemostat that provide for discrete and repeatable adjustments to the flow control element 215.

FIGS. 3A-3D illustrate an interatrial shunting system 300 having an actuation assembly 316 configured in accordance with select embodiments of the present technology. More specifically, FIGS. 3A and 3B are side views of the interatrial shunting system 300 in a first and second configuration, respectively. FIGS. 3C and 3D are enlarged views of the actuation assembly 316 in the first and second configurations, respectively. As will be described in detail below, the system 300 is adjustable through a plurality of discrete geometries, with each geometry providing a different relative flow or drainage resistance and/or flow rate through the system 300. Accordingly, in some embodiments, the system 300 includes a selectively titratable system for allowing the movement of fluid, such as blood flowing between a LA and a RA to treat HF.

Certain aspects of the system 300 can be generally similar to certain aspects of the system 200, described in detail above with respect to FIGS. 2A and 2B. For example, referring to FIGS. 3A and 3B, the system 300 can include a shunting element 302 having a lumen (not shown) extending therethrough. The system 300 can also include a membrane 312 coupled to the shunting element 302 to define an aperture 314 configured to fluidly connect the lumen with the LA or the RA when the system 300 is implanted in a patient. The system 300 further includes the actuation assembly 316 operably coupled to a flow control element 315. The flow control element 315 can be operably coupled to the aperture 314. As described below, actuation of the actuation assembly 316 can adjust a geometry of the flow control element 315, which in turn adjusts a geometry of the aperture 314.

In some embodiments, the flow control element 315 is generally similar to the flow control element 215 described above with respect to FIGS. 2A and 2B. For example, the flow control element 315 can include a string element having a loop portion 320 disposed generally around the aperture 314 and a connecting portion 322 extending between the loop portion 320 and the actuation assembly 316. The loop portion 320 can be loosened or tightened and/or shifted in position, and thus the diameter of the aperture 314 can change, by pulling on or releasing the connecting portion 322, as described in detail above with respect to FIGS. 2A and 2B. In some embodiments, the flow control element 315 may further include a stabilization element 323 that interfaces with the loop portion 320 and/or the connecting portion 322. In some implementations, the stabilization element 323 is a rigid conduit through which at least a section of loop portion 320 and/or connection portion 322 travels.

Referring to FIG. 3A, the system 300 is illustrated in a first configuration in which the aperture 314 has a first diameter D₁. Upon actuation of the actuation assembly 316, as described in detail with reference to FIGS. 3C and 3D, the system 300 can transition from the first configuration (with the aperture 314 having the first diameter D₁) to another configuration with aperture 314 having a different diameter. For example, referring to FIG. 3B, the system 300 is illustrated in a second configuration in which the aperture 314 has a second diameter D₂. Although the second diameter D₂ is illustrated as smaller than the first diameter D₁, in some embodiments the aperture 314 may also be transitionable from a smaller diameter to a greater diameter (e.g., moving from the second configuration to the first configuration). In embodiments, the system 300 is adjustable into a plurality of configurations corresponding to a plurality of aperture diameters and/or a plurality of flow rates for a given patient condition.

FIGS. 3C and 3D illustrate additional features of the actuation assembly 316 that enable the aperture 314 to be adjusted through a plurality of discrete geometries. For example, the actuation assembly 316 can include an actuation component or engine 340 and a ratchet mechanism 330. The actuation component 340 includes an elastic element 342 and an actuation element 344 disposed within the elastic element. The elastic element 342 can comprise any elastic material that can compress, expand, or otherwise deform in response to a force and recoil towards the initial position once the force is removed, such as silicone, natural or synthetic rubbers, blends or combinations of these materials, or other suitable materials. The actuation element 344 can be composed of a shape memory material, such as a shape memory alloy (e.g., nitinol). Accordingly, the actuation element 344 can be transitionable between a first material state (e.g., a martensitic state, a R-phase, etc.) and a second material state (e.g., a R-phase, an austenitic state, etc.). In the first material state, the actuation element 344 may be relatively deformable (e.g., plastic, malleable, compressible, expandable, etc.). In the second material state, the actuation element 344 may have a preference toward a specific geometry (e.g., a heat set geometry, an original geometry, etc.) that has a specific shape, length, and/or other dimension.

The actuation element 344 can be transitioned between the first material state and the second material state by applying energy (e.g., heat) to the actuation element 344 to heat the actuation element 344 above a transition temperature. In some embodiments, the transition temperature for the actuation element 344 is greater than an average body temperature. Accordingly, the actuation element 344 is typically in the first material state when the system 300 is implanted in the body until the actuation element 344 is heated. If the actuation element 344 is deformed relative to its preferred geometry (e.g., the heat set geometry, the original geometry, etc.) while in the first material state, heating the actuation element 344 above its transition temperature causes the actuation element 344 to transition to the second material state and therefore transition from the deformed shape towards the preferred shape. Heat can be applied to the actuation element 344 via RF heating, resistive heating, or other suitable techniques.

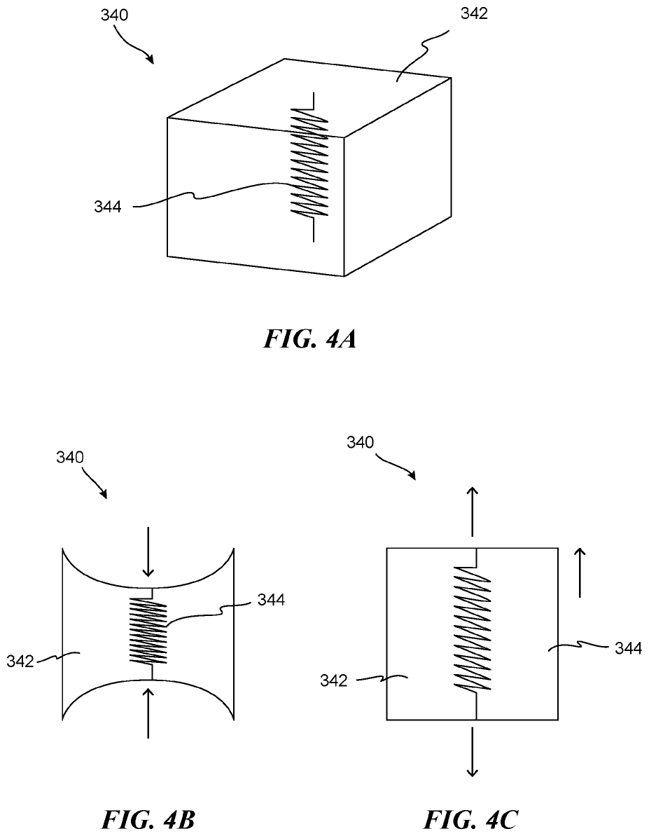

Referring now to FIG. 4A, the actuation component 340 is shown in a first (e.g., neutral) configuration. In the neutral configuration, the actuation element 344 is in the first material state and is lengthened or otherwise deformed relative to its preferred geometry (e.g., a heat set geometry, a shape set geometry, an original geometry, etc.). In the embodiment shown, when the actuation element 344 is in the first material state, it remains relatively malleable and therefore the shape and material properties of the elastic element 342 holds the actuation element 342 in a deformed (e.g., elongated) state (i.e., in a geometry that is deformed from the preferred geometry). Accordingly, because the actuation element 344 is typically in the first material state when the system 300 (FIG. 3A) is at body temperature, the actuation component 340 is typically in the neutral state. However, upon heating the actuation element 344 above its transition temperature to transition it from the first material state (e.g., martensitic) to the second material state (e.g., austenitic), the force driving the actuation component 344 towards its preferred geometry (e.g., the shape set geometry) overcomes the elastic force of the elastic element 342. This causes the actuation element to move towards its preferred geometry by shortening or otherwise compressing, which causes the elastic element 342 to also compress or otherwise deform, as best shown in FIG. 4B. As described below, this contraction of the elastic element 342 leads to an induction of motion in the flow control element 315. Once the heat in the actuation element 344 dissipates and the actuation element 344 falls below its transition temperature, the actuation element 344 returns to the first material state (e.g., martensitic) where it is relatively malleable, and as such the elastic recoil force of the elastic element 342 forces the actuation element 344 away from its preferred geometry (e.g., away from its shape set geometry) and into the neutral configuration, as shown in FIG. 4C.

Returning back to FIGS. 3C and 3D, the actuation assembly 316 also includes the ratchet mechanism 330. The ratchet mechanism 330 includes a plurality of teeth 334 defining a plurality of grooves 335 therebetween. The teeth 334 can have a sawtooth or other suitable configuration, thereby providing a “one-way” ratchet, as described below. In the illustrated embodiment, there are three grooves 335 (a first groove 335a, a second groove 335b, and a third groove 335c), although in other embodiments, more or fewer grooves may be included on the ratchet mechanism 330. As will be appreciated by one skilled in the art in view of the following description, increasing the number of grooves 335 generally increases the number of discrete geometries the aperture 314 (FIGS. 3A and 3B) can assume. The number of grooves 335 can be increased by increasing an overall length of the ratchet mechanism 330 and/or decreasing the spacing between adjacent grooves 335 (e.g., decreasing a width of the teeth 334). Increasing a pitch of the grooves 335 may also generally increase the granularity of potential adjustments to the aperture 314 by allowing for relatively smaller movements of the flow control element 315. The ratchet mechanism 330 may also include a ramp structure 336. As described in detail below, the ramp structure 336 may enable the system 300 to function similar to a hemostat and allows the actuation assembly 316 to be “reset” following a predetermined number of actuations.

The actuation assembly 316 further includes an engagement member 324 coupled to the connecting portion 322 of the flow control element 315. For example, as the connecting portion 322 is drawn towards the actuation component 340 via actuation of the actuation component 340, the engagement member 324 is also drawn towards the actuation component 340. The engagement member 324 is configured to interface with or otherwise engage the ratchet mechanism 330. For example, the engagement member 324 can be a hook or other “L” shaped structure that can engage with one of the grooves 335 defined by the teeth 334. For example, referring now to FIG. 3C, the actuation assembly 316 is shown in a first configuration in which the engagement member 324 is engaged with the ratchet mechanism 330 at the first groove 335a. The connecting portion 322 therefore extends between the actuation assembly 316 and the loop portion 320, and is operably coupled to the ratchet mechanism 330 via the engagement member 324. When the actuation element 344 is actuated, and as described above with respect to FIGS. 4A-4C, the actuation component 240 transitions from the neutral configuration (FIG. 4A) to the compressed configuration (FIG. 4B). Because the actuation component 340 is connected to the connecting portion 322, transitioning the actuation component 240 to the compressed configuration pulls the connecting portion 322 and engagement member 324 towards the actuation component 340. This has two primary effects. First, it causes the engagement member 324 to move from the first groove 335a to the second groove 335b. Second, it also causes the connecting portion 322 to tighten the loop portion 320 of the flow control element 315, thereby decreasing a diameter of the aperture 314.

When the actuation element 344 cools below its transition temperature, the actuation component 340 returns to the neutral configuration (FIG. 4C). However, as noted above the ratchet mechanism 330 can be a “one-way” ratchet that, in most configurations, primarily permits movement of the engagement member 324 in a single direction (i.e., towards the actuation component 340), such that the engagement member 324, and thus the connecting portion 322, do not move back towards its pre-actuated position as the actuation component 340 returns to the neutral configuration. This means the flow control element 315 remains in its adjusted position following actuation and the aperture 314 retains its decreased diameter.

The ratchet mechanism 330 can limit movement of the engagement member 324 to be primarily in a single direction through any number of suitable techniques. For example, the teeth 334 can have a generally sawtooth configuration such that the engagement member 324 can move from the first groove 335a to the second groove 335b (e.g., by sliding up the inclined/sloped surface of a tooth 334), but not vice versa, as the flat backside of the teeth 334 will interface with the engagement member 324 and limit movement in the opposing direction. Likewise, the engagement member 324 can move from the second groove 335b to the third groove 335c, but not vice versa. In embodiments with a “one-way” ratchet mechanism, such as the illustrated embodiment, the ratchet mechanism can include a “reset” in which the ratchet mechanism returns the engagement member 324 to the first groove 335a. In some embodiments, this reset may function in a manner similar to a hemostat device. For example, in the illustrated embodiment, the ratchet mechanism 330 includes a ramp structure 336. Once the engagement member 324 is in the groove closest to the actuation component 340 (the third groove 335c in the illustrated embodiment), further actuation of the actuation element 344 moves the engagement member 324 out of the grooves 335 and onto the ramp structure 336, which directs the engagement member 324 back to the first groove 335a, thereby resetting the actuation assembly 316.

In the embodiment shown, the net effect of moving the engagement member 324 from the first groove 335a to the second groove 335b is transitioning the system from a first configuration in which the aperture 314 has a first size (e.g., a first diameter) (e.g., FIG. 3A) to a second configuration in which the aperture 314 has a second size (e.g., a second diameter) that is less than the first size (e.g., FIG. 3B). As a result of the ratchet mechanism 330, the system 300 is configured to retain the second configuration having the second size even as the actuation component 340 returns to its neutral configuration. The actuation assembly 316 can then be actuated again to move the engagement member 324 from the second groove 335b to the third groove 335c (FIG. 3D), causing the system to transition to a third configuration in which the aperture 314 has a third size that is less than the second size. Once again, the system 300 is configured to retain the third configuration having the third size even as the actuation component 340 returns to its neutral configuration. The actuation assembly 316 can then be actuated again to move the engagement member 324 from the third groove 335c back to the first groove 335a via the ramp structure 336, thereby transitioning the system from the third configuration having the third size to the first configuration having the first size. Accordingly, actuating the actuation assembly 316 to move the engagement member 324 can selectively and discretely adjust the aperture 314 through a plurality of geometries. Each geometry can impart a different relative flow resistance and/or flow of fluid through the shunting element 302 and aperture 314, providing a plurality of different therapy levels. For example, when the aperture 314 has the first diameter (FIG. 3A), the shunting element may have a first relative flow resistance. When the aperture 314 has the second diameter (FIG. 3B) that is less than the first diameter, the shunting element 302 can have a second relative flow resistance that is greater than the first relative flow resistance. Accordingly, in some embodiments moving the system 300 from the first configuration to the second configuration can decrease flow between the LA and the RA. As provided above, the number of discrete geometries is determined based on, for example, the number of grooves 335 in the ratchet mechanism. In variation embodiments, the system 300 may have the opposite relationship between the ratchet mechanism and aperture size as described above (i.e., the system 300 may be configured such that actuating the actuating assembly 316 to move the engagement member 324 closer to the actuation component 340 will result in an increase of size of the aperture 314).

As one skilled in the art will appreciate, the actuation assembly 316 can be adapted for use with other adjustable shunts, including other adjustable interatrial shunts. For example, the actuation assembly 316 can be used to control the movement of flow control elements beyond those expressly described herein. Therefore, the present technology is not limited to the embodiments described herein, and instead provides a mechanism for discretely and systematically adjusting a medical device, which in turn enables the medical device to provide a titratable therapy.

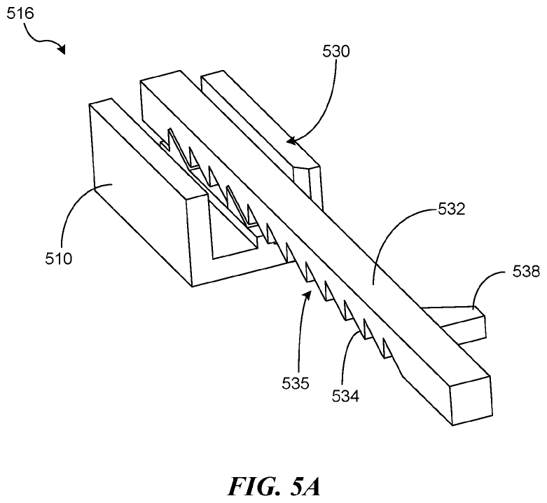

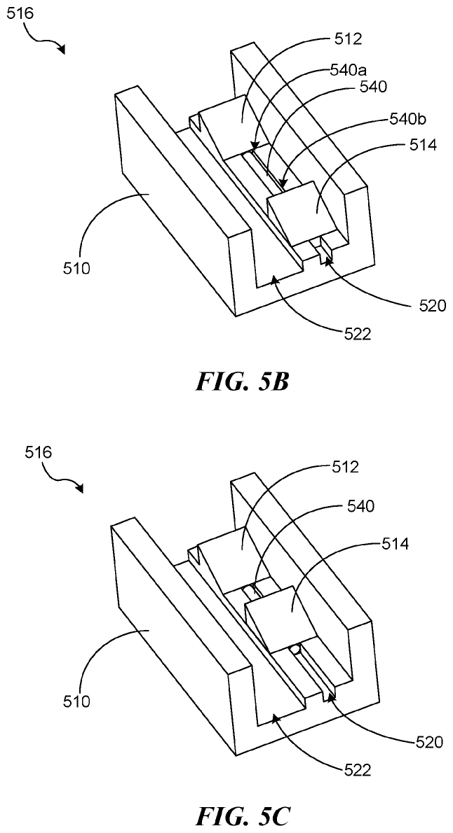

FIGS. 5A-5C illustrate an actuation assembly 516 configured in accordance with select embodiments of the present technology. In some embodiments, the actuation assembly 516 can be used with the interatrial shunting systems 200 or 300 described herein (e.g., instead of actuation assemblies 216 and 316, respectively). In other embodiments, the actuation assembly 516 can be used with other suitable adjustable interatrial shunting systems. As will be described in detail below, the actuation assembly 516 provides another mechanism for selectively transitioning an adjustable shunt between a plurality of discrete geometries, with each geometry providing a different relative flow or drainage resistance and/or flow rate.

Referring to FIG. 5A, the actuation assembly 516 includes a housing structure 510 and a ratchet mechanism 530. The ratchet mechanism 530 includes a rack element 532 having a plurality of teeth 534 and a plurality of grooves 535 defined between the plurality of teeth 534. The rack element 532 can further include a reset feature 538 (e.g., a projection, knob, etc.). In some embodiments, the rack element 532 can be operably coupled to a flow control element (e.g., flow control element 315 on system 300, shown in FIGS. 3A-3B—no flow control element is shown in FIG. 5A). As described in detail below with respect to FIGS. 5B and 5C, the rack element 532 is moveable through a plurality of discrete positions relative to the housing 510. Moving the rack element 532 through the plurality of discrete positions relative to the housing 510 can move the flow control element through a plurality of corresponding discrete geometries, therefore adjusting the shunt (not shown).

FIG. 5B illustrates the actuation assembly 510 with the rack element 532 omitted for purposes of clarity. As shown, the housing 510 can include a first engagement member 512 and a second engagement member 514. In some embodiments, the first engagement member 512 and the second engagement member 514 can be first and second pawls, respectively. The first engagement member 512 can be connected to and/or integral with the housing 510 such that it does not move with respect to the housing 510. The second engagement member 514 can be coupled to the housing 510 such that it is moveable with respect to the housing 510. For example, the housing 510 can include a track 520 (e.g., a recess, a channel, etc.) configured to receive at least a portion of the second engagement member 514. In some embodiments, the track 520 can permit movement of the second engagement member 514 in a single dimension or plane of motion, while limiting movement in other dimensions or planes of motion. The first engagement member 512 can be configured to engage with a groove 535 (e.g., a first groove) on the rack element 532 (FIG. 5A). Likewise, the second engagement member 514 can be configured to engage a groove 535 (e.g., a second groove) on the rack element 532.

The actuation assembly 516 can further include an actuation component 540 operably coupled to and configured to move the second engagement member 514 with respect to the housing 510. In some embodiments, for example, the actuation component 540 is positioned within the track 520 between the first engagement member 512 and the second engagement member 514. A first end portion 540a of the actuation component 540 can be secured to the housing 510 (e.g., secured to the first engagement member 512). A second end portion 540b of the actuation component 540 can be secured to the second engagement member 514. The actuation component 540 can include an elastic element (not shown) and an actuation element (e.g., a shape memory wire—not shown). The elastic element can comprise any elastic material that can compress, expand, or otherwise deform in response to a force and recoil towards the initial position once the force is removed, such as silicone, natural or synthetic rubbers, blends or combinations of these materials, or other suitable elastic materials (e.g., a spring). The actuation element can comprise a shape memory alloy (e.g., nitinol). Accordingly, the actuation element can be transitionable between a first material state (e.g., a martensitic state, a R-phase, etc.) and a second material state (e.g., a R-phase, an austenitic state, etc.). In the first material state, the actuation element may be relatively deformable (e.g., plastic, malleable, compressible, expandable, etc.). In the second material state, the actuation element may have a preference toward a specific geometry (e.g., a heat set geometry, a shape set geometry, an original geometry, etc.) that has a specific shape, length, and/or other dimension.

The actuation element can be transitioned between the first material state and the second material state by applying energy (e.g., heat) to the actuation component 540 to heat the actuation element above a transition temperature. In some embodiments, the transition temperature for the actuation element is greater than an average body temperature. Accordingly, the actuation element is typically in the first material state when implanted in the body until the actuation component 540 is heated. If the actuation element is deformed relative to its preferred geometry while in the first material state, heating the actuation component 540 above its transition temperature causes the actuation element to transition to the second material state and therefore move towards its preferred geometry. Heat can be applied to the actuation component 540 via RF heating, resistive heating, or other suitable techniques.

In some embodiments, the elastic element and the actuation element can operate in a similar manner as the elastic element 342 and the actuation element 344 described above with respect to FIGS. 4A-4C. In FIG. 5B, for example, the actuation component 540 is shown in a first (e.g., neutral) configuration. In the neutral configuration, the actuation element is in the first material state and is lengthened or otherwise deformed relative to its preferred geometry (e.g., the heat set geometry, the shape set geometry, the original geometry, etc.). In the embodiment shown, when the actuation element is in the first material state, it remains relatively malleable and therefore the shape and material properties of the elastic element holds the actuation element in the deformed (e.g., elongated) state. Accordingly, because the actuation element is typically in the first material state when at body temperature, the actuation component 540 is typically in the neutral state. However, upon heating the actuation component 540 above the actuation element's transition temperature to transition the actuation element from the first material state (e.g., martensitic) to the second material state (e.g., austenitic), the force driving the actuation element towards its preferred geometry overcomes the elastic force of the elastic element. This causes the actuation element to move towards its preferred geometry by shortening or otherwise compressing, which causes the elastic element to also compress or otherwise deform. In some embodiments, the net effect of this transition is moving at least one aspect of actuation component 540 closer to the first engagement member 512 (e.g., via the shortening of the actuation component 540, as best shown in FIG. 5C). Because the second end portion 540b of the actuation component 540 is coupled to the second engagement member 514, the second engagement member 514 is pulled towards the first engagement member 512 when the actuation component 540 is shortened. Once the heat in the actuation component 540 dissipates and the actuation element falls below its transition temperature, the actuation element returns to the first material state (e.g., martensitic) where it is relatively malleable, and as such the elastic recoil force of the elastic element forces the actuation element away from its preferred geometry (e.g., away from its shape set geometry) and back into the neutral configuration, as shown in FIG. 5A.

Referring now to FIGS. 5A-5C together, when the rack element 532 is coupled to the first engagement member 512 and the second engagement member 514, actuation of the actuation component 540 (e.g., transitioning from the neutral configuration shown in FIG. 5B to the actuated configuration shown in FIG. 5C) pulls the rack element in a first direction (e.g., further towards the first engagement member 512). More specifically, as the second engagement member 514 moves towards the first engagement member 512 during actuation of the actuation component 540, the second engagement member 514 remains within the same groove 535 on the rack element 532 while the first engagement member 512 slides down one groove 535 on the rack element 532. This occurs because, as the second engagement member 514 moves towards the first engagement member 512, the second engagement member 514 engages a flat surface of a tooth 534 whereas the first engagement member 512 engages an inclined or otherwise sloped surface of a tooth 534. If the rack element 532 is coupled to a flow control element (not shown), this motion can induce a geometry change in the flow control element.

When the actuation component 540 transitions from the actuated configuration (FIG. 5C) back to the neutral configuration (FIG. 5B)—causing the second engagement member 514 to move away from the first engagement member 512—the first engagement member 512 remains in the same groove 535 on the rack element 532 while the second engagement member 514 slides down one groove 535 on the rack element 532. This occurs because, as the second engagement member 514 moves away from the first engagement member 512, the first engagement member 512 now engages a flat surface of a tooth 534 whereas the second engagement member 514 now engages an inclined or otherwise sloped surface of a tooth 534. As a result, the rack element 532 does not move in a second direction opposite the first direction as the actuation component 540 resets from the actuated configuration (FIG. 5C) to the neutral configuration (FIG. 5B). The net effect of the foregoing operation is movement of the rack element primarily in the first direction, which, as described in detail with respect to FIGS. 3A-4C, can impart a discrete and retainable geometry change in a flow control element.

The actuation assembly 516 can also include a “reset” in which the rack element 532 returns to an original position (e.g., such as shown in FIG. 5A) once it has reached the end of its possible movement in the first direction (e.g., when a distalmost groove 335 engages the second engagement member 514). In some embodiments, this reset may function in a manner similar to a hemostat device. For example, in the illustrated embodiment, the housing 510 includes a return channel or ramp structure 522. Once the engagement member 324 is in the groove closest to the actuation component 340 (the third groove 335c in the illustrated embodiment), further actuation of the actuation component 540 moves the rack element 532 out of engagement with the first engagement member 512 and the second engagement member 514 and onto the return channel 522, which directs the rack element 532 back to its original position, thereby resetting the actuation assembly 516. In some embodiments, the reset feature 538 can direct the rack element 532 into the return channel 522 by, for example, interacting with a portion of the housing 510. When the rack element 532 is connected to a flow control element, movement of the rack element 532 along the return channel 522 can return the flow control element to an initial geometry.

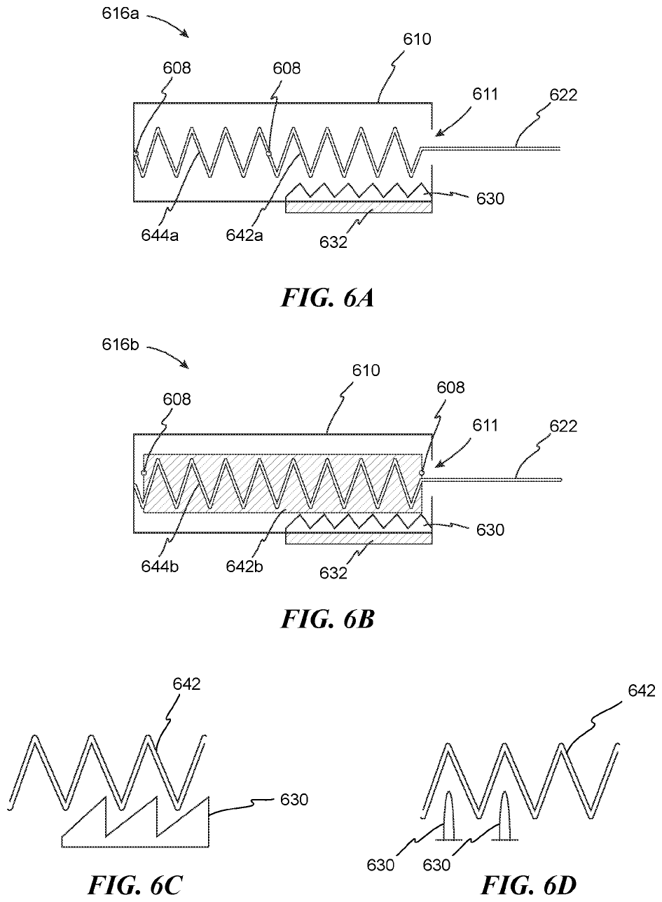

FIGS. 6A-6D illustrate additional actuation assemblies 616a and 616b configured in accordance with select embodiments of the present technology. In some embodiments, the actuation assemblies 616a and 616b can be used with the interatrial shunting systems 200 or 300 described herein (e.g., instead of actuation assemblies 216 and 316, respectively). In other embodiments, the actuation assemblies 616a, 616b can be used with other suitable adjustable interatrial shunting systems. As will be described in detail below, the actuation assemblies 616a and 616b provide yet another mechanism for selectively transitioning an adjustable shunt between a plurality of discrete geometries, with each geometry providing a different relative flow or drainage resistance and/or flow rate.

Referring first to FIG. 6A, the actuation assembly 616a can include a housing 610 (e.g., a rigid enclosure) and an actuation element 644a and an elastic element 642a (e.g., a counterbalance element) carried by the housing 610. The actuation element 644a can be composed of a thermo-elastic and/or shape memory material (e.g., Nitinol) that is relatively malleable at room and body temperature owing to the fact that a transformation temperature (e.g., Rs, As, Rf, Af) is above body temperature. The elastic element 642a can be composed of an elastic-plastic material (e.g., stainless steel, silicone, urethane, etc.).

In some embodiments, the actuation element 644a and the elastic element 642a can both be formed in a spring-like shape. In its most basic form, a spring can be characterized by the equation by F=k(x₁-x₀), where F is the force stored in a spring that has been deflected from its initial position x0 to another position x1. The spring constant, k, is governed by the spring's cross-sectional geometry, pitch diameter, number of coils, and underlying material properties (e.g., elastic modulus, plateau stress, etc.). In some embodiments, the choice of materials for both the actuation element 644a and the elastic element 642a can be selected such that kₐ₁<k_{c}<kₐ₂; where kₐ₁ is the actuation element's spring constant at a body temperature, k_{c} is the elastic element's spring constant at body temperature, and kₐ₂ is the actuation element's spring constant at the temperature above body temperature to which the actuation element is heated to drive movement. The mechanism of kₐ₂>kₐ₁ is due to a partial or full phase transformation from a relatively malleable state (e.g., martensitic) to a relatively stiff state (austenitic), such as described above with respect to actuation component 340 (FIGS. 3A-4C). The actuation element 644a and elastic element 642a can have as-manufactured lengths of Lₐ and L_{c}, respectively. The housing 610 can have an inner dimension, Lₑ, within which the actuation element 644a and the elastic element 642a are positioned, such that (Lₐ+L_{c})≠Lₑ. Accordingly, the actuation element 644a and/or the elastic element 642a need to be compressed or extended to be installed into the housing 610. This compression or extension stores residual energy in one, or both, of the actuation element 644a and/or the elastic element 642a. For example, in the case where (Lₐ+Lₑ)>Lₑ, a force F1 is applied to compress the actuation element 644a and/or the elastic element 642a to position the same within the housing 610. Because the actuation element 644a and the elastic element 642a are joined in series, they experience the same applied force. And because the spring constant of the actuation element 644a when in the first material state is less than the spring constant of the elastic element 642a (e.g., kₐ₁<k_{c}), the actuation element 644a is compressed more than the elastic element 642a.

The actuation element 644a and/or the elastic element 642a can be connected to a flow control element (not shown) via a connecting line 622. For example, in embodiments in which the actuation assembly 616a is used in connection with the system 200 (FIGS. 2A-2B), the actuation assembly may be set such that the flow control element 215 is at its largest geometry (e.g., largest diameter) initially. To decrease the diameter of the flow control element 215, the actuation element 644a is heated using, for example, an electrical lead 608. In its heated condition, the force in the actuation element 644a rises to F2, where F2>F1, due to the fact that kₐ₁<kₐ₂ (e.g., by transforming from a martensitic material state to an austenitic material state). Because k_{c}<kₐ₂, the elevated force from the heated actuation element 644a is sufficient enough to move the elastic element, pulling the connecting line 622 into the housing 610 and thereby decreasing the diameter of the flow control element 215. In other embodiments, the actuation assembly 616a can have the opposite relationship with the flow control element such that actuating the actuation element 644a moves the flow control element from a smaller geometry to a larger geometry.

If nothing else was done other than removing the heat, the actuation assembly 616a would return to its original position once the spring constant of the actuation element 644a returned to kₐ₁ (e.g., once the actuation element 644a cooled below its transition temperature and returned to the first material state). However, the actuation assembly 616a can optionally include a locking mechanism 630. The locking mechanism 630 can be activated when the actuation element 644a is heated such that the adjustment to the flow control element (not shown) is retained once the actuation element 644a cools below its transition temperature. Consequently, when the actuation element 644a cools below its transition temperature, the stored energy in the elastic element 642a is transferred to the locking mechanism 630 rather than to the actuation element 644a. The locking mechanism 630 may therefore control the relative position of the flow control element. The locking mechanism 630 may be any suitable locking mechanism. For example, as illustrated in FIG. 6C, the locking mechanism 630 may comprise a one-way rack having a plurality of teeth. In another example, and as shown in FIG. 6D, the locking mechanism 630 may comprise a plurality of pins. In yet other embodiments, the locking mechanism 630 may comprise a ratchet mechanism, such as those previously described herein. Regardless of its configuration, the locking mechanism 630 can also include a release element 632 configured to “release” the locking mechanism 630. When released, the locking mechanism 630 and the elastic element 642a disengage, thereby releasing the elastic element's stored energy into the actuation element 644a and driving the actuation assembly 616a (and the flow control element) back to the original configuration (shown in FIG. 6A).

In some embodiments, the locking mechanism 630 can engage other aspects of the actuation assembly 616a instead of, or in addition to, the elastic element 642a. For example, in some embodiments the locking mechanism 630 may engage the actuation element 644a. In yet other embodiments, the locking mechanism 630 can be generally similar to the ratchet mechanism 330 described with respect to FIGS. 3A-3D and be configured to engage the connecting line 622. Accordingly, in some embodiments, the spring-like engine (e.g., the actuation element 644a and the elastic element 642a) of the actuation assembly 616a can be used with the system 300 instead of the actuation component 340.

In some embodiments, the orientation of the actuation element 644a and the elastic element 642a can be reversed, such that the actuation element 644a is coupled to the connecting line 622. In some embodiments, multiple actuation elements 644a and elastic elements 642a can be arranged in series and/or in parallel. In such embodiments, the actuation assembly 616a may also include multiple individually-activatable locking mechanisms 630. Incorporating multiple, individually actuatable actuation element 644a could provide greater granularity of adjustments to a flow control element coupled to the actuation assembly 616a. If arranged in series, the overall height and/or width of the housing 610 could remain generally the same while the length of the housing 610 would be increased. If arranged in parallel, the overall length of the housing 610 could remain generally the same but the height and/or width of the housing 610 would be increased.

FIG. 6B illustrates another actuation assembly 616b. The actuation assembly 616b can be generally similar to the actuation assembly 616a, except that actuation element 644b is disposed within elastic element 642b. For example, the actuation assembly 616b can operate in the same, or substantially the same, manner as the actuation component 340 described with respect to FIGS. 4A-4C. Without being bound by theory, the configuration shown in FIG. 6B is expected to reduce the amount of heat that leaks out of the actuation assembly 616b and into the surrounding tissue. For example, because the heated component (the actuation element 644b) is disposed within the elastic element 642b, heat from the actuation element 644b is absorbed by the elastic element 642b and does not spread (or spreads to a lesser extent) into the tissue surrounding the actuation assembly 616b. Accordingly, in some embodiments, the actuation element 644b can be heated to a higher temperature without causing unwanted tissue heating.

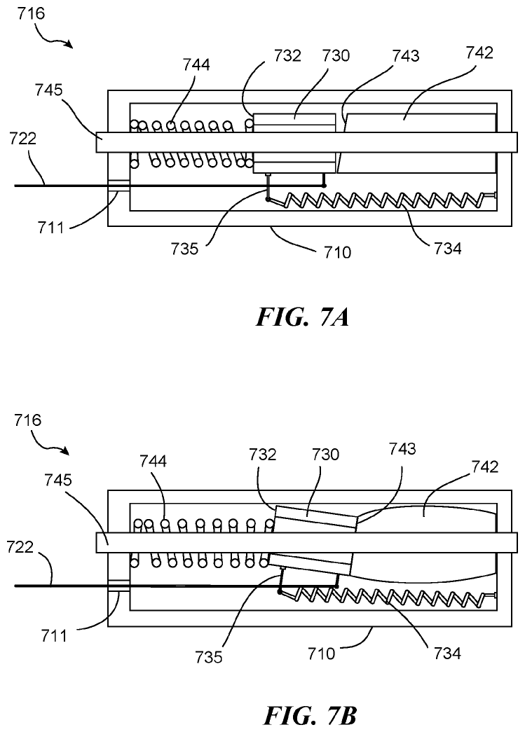

FIGS. 7A and 7B illustrate yet another actuation assembly 716 configured in accordance with select embodiments of the present technology. In some embodiments, the actuation assembly 716 can be used with the interatrial shunting systems 200 or 300 described herein (e.g., instead of actuation assemblies 216 and 316, respectively). In other embodiments, the actuation assembly 716 can be used with other suitable adjustable interatrial shunting systems. As will be described in detail below, the actuation assembly 716 provides yet another mechanism for selectively transitioning an adjustable shunt between a plurality of discrete geometries, with each geometry providing a different relative flow or drainage resistance and/or flow rate.

The actuation assembly 716 includes a cam-lock type mechanism. More specifically, the actuation assembly 716 includes a housing 710 having an opening 711 for receiving a portion of a connecting line 722. In some embodiments, the connecting line 722 can be the same as, or generally similar to, the connecting line 222 described above with respect to FIGS. 2A and 2B. Accordingly, in some embodiments, the connecting line 722 can be connected to a flow control element (not shown) configured to adjust a geometry of a shunt. The actuation assembly 716 can further include an elongated rod-like shaft element 745 extending from a first end portion of the housing 710 to a second end potion of the housing 710. In some embodiments, the shaft element 745 is coupled to the housing 710 such that it does not move with respect to the housing 710.

The actuation assembly 716 can further include an actuation element 744, an elastic element 742, and a locking mechanism 730 positioned between the actuation element 744 and the elastic element 742. As previously described in detail with respect to other embodiments, the actuation element 744 can be composed of a shape memory material and the elastic element 742 can be composed of any suitable elastic material. The actuation element 744, the elastic element 742, and/or the locking mechanism 730 may be positioned around the shaft element 745. For example, the actuation element 744 can have a helical arrangement, with the shaft element 745 extending through a center of the helix. The locking mechanism 730 and/or the elastic element 742 can have a tube-like design such that the shaft element 745 can extend through a central lumen(s) of the locking mechanism 730 and/or the elastic element 742. In some embodiments, the locking mechanism 730 can have a hardened knife-like edge 732 that, as described below with respect to FIG. 7B, can form a friction interface with the shaft element 745. The elastic element 742 can have an angled face 743 configured to engage with a portion of the locking mechanism 730 (e.g., the portion of the locking mechanism 730 opposite from the edge 732). In some embodiments, the actuation assembly 716 can further include a release element 734. The release element 734 may also be composed of a shape memory material and can be operably coupled to the locking mechanism 730 via a connecting element 735 (e.g., a line, string, chain, or the like).

FIG. 7A shows actuation assembly 716 in a relaxed or neutral (e.g., pre-tensioned and/or pre-actuated) configuration, in order to show the angled face 743 of the elastic element 742. Both the actuation element 744 and the release element 734 are in a first material state (e.g., a martensitic material state) at body temperature such that they can be deformed relative to their preferred geometry (e.g., a heat set geometry, a shape set geometry, an original geometry, etc.). In the neutral (pre-actuated) configuration, the actuation element 744 is compressed relative to its preferred geometry. In order to place tension on the connecting line 722 (thus changing a geometry of a flow control element coupled to the connecting line 722), the actuation element 744 is heated above its transition temperature such that it transitions from the first material state to a second material state (e.g., an austenitic material state). Upon heating the actuation element 744 above the transition temperature to transition the actuation element 744 from the first material state (e.g., martensitic) to the second material state (e.g., austenitic), the force driving the actuation element 744 towards its preferred geometry overcomes the elastic force of the elastic element 742. This causes the actuation element 744 to move towards its preferred geometry by expanding or otherwise lengthening, which pushes the locking mechanism 730 towards the elastic element 742 and causes the elastic element 742 to compress or otherwise deform. However, because the locking mechanism 730 engages the angled face 743 on the elastic element 742 as the actuation element 744 expands, the force exerted on the elastic element 742 by the locking mechanism 730 is “off-axis” (e.g., angled relative to the longitudinal axis of the shaft element 745). For example, as best shown in FIG. 7B, actuation of the actuation element 744 drives the locking mechanism 730 into an angled orientation relative to a longitudinal axis of the shaft element 745. In addition, because the release element 734 is coupled to the locking mechanism 730 via the connecting element 735 and is in the first material state (e.g., the martensitic material state), the release element 734 is deformed (e.g., compressed) relative to its preferred geometry as the actuation element 744 transitions towards its preferred geometry.

When the actuation element 744 cools below its transition temperature such that the elastic counterforce of the elastic element 742 overcomes the force pushing the actuation element 744 towards its preferred geometry, the “off-axis” force generated by the interface between the locking mechanism 730 and the angled face 743 of the elastic element 742 causes the edge 732 of the locking mechanism 730 to dig into or otherwise interface with a roughened surface of the shaft element 745, keeping the locking mechanism 730 (and thus the actuation element 744) in the actuated configuration (e.g., the configuration shown in FIG. 7B). Additional force can be created by having the connecting line 722 located on the same side as the longer edge of the elastic element 742, thus providing more off-axis locking force.

To disengage the locking mechanism 730 and return the actuation assembly 716 to its original (e.g., pre-actuated) configuration, the release element 734 can be heated above its transition temperature such that it transitions from the first material state (e.g., the martensitic material state) to the second material state (e.g., the austenitic material state). Because the release element 734 was compressed relative to its preferred geometry during actuation of the actuation element 744, heating the release element 734 above its transition temperature increases the force driving the release element 734 towards its preferred (e.g., lengthened) geometry. This force, which is generally parallel to the longitudinal axis of the shaft element 745, disengages the edge 732 of the locking mechanism 730 from the shaft element 745. To do so, the force generated by heating the release element 734 should be at least momentarily greater than the force stored in the elastic element 742 that is pushing the edge 732 into the shaft element 745. This allows the actuation assembly to return to and/or toward its pre-actuated configuration, shown in FIG. 7A.

As one skilled in the art will appreciate, various features of the present technology described herein can be combined to form shunting systems not explicitly described herein. For example, any of the actuation assemblies described herein can be adapted for use with the system 200 or the system 300, or another suitable interatrial shunting system. In another example, in some embodiments one or more portions of one actuation assembly or device described herein can be combined with one or more portions of another actuation assembly or device described herein. Accordingly, the present technology is not limited to the embodiments explicitly illustrated and discussed herein.

In embodiments of the present technology that utilize heat or another form of energy applied to a shape memory element or another component of the system, the energy/heat can be applied both invasively (e.g., via a catheter delivering laser, radiofrequency, or another form of energy, via an internal stored energy source such as a supercapacitor, etc.), non-invasively (e.g., using radiofrequency energy delivered by a transmitter outside of the body, by focused ultrasound, etc.), or through a combination of these methods.

The present technology enables a heart failure treatment to be adjusted over a period of time to provide a more effective therapy. Some embodiments of the present technology adjust the geometry of the shunt (e.g., the diameter of the aperture 314) consistently (e.g., continuously, hourly, daily, etc.). Consistent adjustments might be made, for example, to adjust the flow of blood based on a blood pressure level, respiratory rate, heart rate, and/or another parameter of the patient, which changes frequently over the course of a day. In some embodiments, for example, consistent adjustments can be made based on, or in response to, physiological parameters that are detected using sensors, including, for example, sensed left atrial pressure and/or right atrial pressure. For example, if the left atrial pressure increases, the systems described herein may automatically increase a diameter of the aperture to decrease flow resistance between the LA and the RA and allow increased blood flow. In another example, the systems described herein can be configured to adjust based on, or in response to, an input parameter from another device such as a pulmonary arterial pressure sensor, insertable cardiac monitor, pacemaker, defibrillator, cardioverter, wearable, external ECG or PPG, and the like. Some embodiments of the present technology adjust the geometry of the shunt only after a threshold has been reached (e.g., a sufficient period of time has elapsed). This may be done, for example, to avoid unnecessary back and forth adjustments and/or avoid changes based on clinically insignificant changes.

The present technology also enables a clinician to periodically (e.g., monthly, bi-monthly, annually, as needed, etc.) adjust the geometry of the shunt (e.g., the diameter of the aperture 314) to improve patient treatment. For example, during a patient visit, the clinician can assess a number of patient parameters and determine whether adjusting the diameter of the aperture 314, and thus altering blood flow between the LA and the RA, would provide better treatment and/or enhance the patient's quality of life. Patient parameters can include, for example, physiological parameters (e.g., left atrial blood pressure, right atrial blood pressure, the difference between left atrial blood pressure and right atrial blood pressure, flow velocity, heart rate, cardiac output, myocardial strain, etc.), subjective parameters (e.g., whether the patient is fatigued, how the patient feels during exercise, etc.), and other parameters known in the art for assessing whether a treatment is working. If the clinician decides to adjust the diameter of the aperture 314, the clinician can adjust the system 300 using the techniques described herein.

As one of skill in the art will appreciate from the disclosure herein, various components of the interatrial shunting systems described above can be omitted without deviating from the scope of the present technology. Likewise, additional components not explicitly described above may be added to the interatrial shunting systems without deviating from the scope of the present technology. Accordingly, the systems described herein are not limited to those configurations expressly identified, but rather encompasses variations and alterations of the described systems. Moreover, the following paragraphs provide additional description of various aspects of the present technology. One skilled in the art will appreciate that the following aspects can be incorporated into any of the systems described above.

### EXAMPLES

Several aspects of the present technology are set forth in the following examples:

1. A system for shunting fluid between a first body region and a second body region of a patient, the system comprising:
a shunting element having a lumen extending therethrough and configured to fluidly connect the first body region and the second body region when implanted in the patient; and
a flow control element moveable through a plurality of discrete geometries, wherein each discrete geometry is associated with a relative drainage resistance through the lumen, and wherein the flow control element is selectively moveable between the plurality of discrete geometries.

2. The device of example 1, further comprising an actuation assembly configured to selectively move the flow control element through the plurality of discrete geometries, wherein the actuation assembly includes at least one actuation element and a ratchet mechanism.

3. The device of example 2 wherein the actuation element and the ratchet mechanism are configured to provide a lock step adjustment to the flow control element to move the flow control element through the plurality of discrete geometries.

4. The device of example 2 or 3 wherein the actuation assembly further includes an engagement member operably coupled to the flow control element and the actuation element, and wherein the engagement member is configured to engage the ratchet mechanism.

5. The device of example 4 wherein the ratchet mechanism includes a plurality of teeth defining a plurality of grooves therebetween, and wherein the engagement member engages the ratchet mechanism in one or more of the grooves.

6. The device of example 5 wherein the actuation element is actuatable between a neutral configuration and an actuated configuration, and wherein, when actuated between the neutral configuration and the actuated configuration, (i) the flow control element moves from a first geometry to a second geometry, and (ii) the engagement member moves from a first groove to a second groove.

7. The device of example 6 wherein, when the actuation element moves from the actuated configuration to the neutral configuration, the flow control element retains the second geometry and the engagement member remains in the second groove.

8. The device of any of examples 2-7 wherein the ratchet mechanism has a sawtooth configuration.

9. The device of any of examples 2-8 wherein the ratchet mechanism is a one-way ratchet mechanism that is configured to provide the discrete adjustments to the flow control element geometry in a first direction but prevent adjustment to the flow control element in a second direction opposite the first direction.

10. The device of example 9 wherein the geometry is a diameter, and wherein the discrete adjustments to the flow control element geometry in a first direction comprises making the diameter smaller.

11. The device of example 9 or 10 wherein the ratchet mechanism includes a ramp structure configure to reset the actuation assembly.

12. The device of any of examples 2-11 wherein the actuation element comprises a shape memory material.

13. A system for shunting fluid between a first body region and a second body region of a patient, the system comprising:
a shunting element having a lumen extending therethrough and configured to fluidly connect the first body region and the second body region when implanted within the patient, wherein the shunting element includes an adjustable aperture for controlling flow of fluid through the lumen; and
a flow control element moveable through a plurality of discrete positions, wherein each discrete position is associated with a different aperture geometry, and wherein the flow control element is selectively moveable between the plurality of discrete positions.

14. The system of example 13 wherein the aperture geometry is an aperture diameter.

15. The system of example 13 or 14, further comprising a ratchet mechanism that controls the movement of the flow control element through the plurality of discrete positions.

16. The system of example 15 wherein the ratchet mechanism is configured to selectively decrease the diameter of the aperture while preventing an increase in the diameter of the aperture.

17. The system of example 16 wherein the aperture is moveable between a plurality of diameters, with each corresponding diameter smaller than the previous.

18. A device for treating heart failure, the device comprising:
a lumen configured to fluidly connect a left atrium and a right atrium of a heart of a subject;
a flow control element operably coupled to the lumen; and
an actuation assembly configured to alter the flow of fluid through the lumen by adjusting a geometry of the flow control element, wherein the actuation assembly includes—
   an actuation element,
   a ratchet mechanism having a plurality of grooves, and
   an engagement member operably coupled to the actuation element and the flow control element, wherein the engagement member is configured to engage the ratchet mechanism in one or more of the grooves,
wherein actuation of the actuation element causes (i) the flow control element to move from a first geometry to a second geometry, thereby adjusting the flow of fluid through the lumen, and (ii) the engagement member to move from a first groove to a second groove, thereby maintaining the flow control element in the second geometry.

19. An actuation assembly for use with an adjustable interatrial shunt, the actuation assembly comprising:
an elastic element having a first geometry, and
an actuation element coupled to the elastic element, wherein the actuation element is transitionable between a first material state and a second material state,
wherein transitioning the actuation element from the first material state to the second material state causes the actuation assembly to transition between (i) a pre-actuated configuration in which the actuation element is deformed relative to its preferred geometry, and (ii) an actuated configuration in which the actuation element is closer to its preferred geometry and the elastic element is deformed relative to its first geometry.

20. The actuation assembly of example 19 wherein the actuation assembly is configured to retain the actuated configuration when the actuation element transitions from the second material state to the first material state.

21. The actuation assembly of example 19 or 20, further comprising a locking mechanism, wherein the locking mechanism is configured to engage the elastic element and/or the actuation element to retain the actuation assembly in the actuated configuration.

22. The actuation assembly of example 19 wherein the actuation assembly is configured to return to the pre-actuated configuration when the actuation element transitions from the second material state to the first material state.

23. The actuation assembly of example 22, further comprising a ratchet mechanism operably coupled to the elastic element and/or the actuation element.

24. The actuation assembly of any of examples 19-23 wherein the elastic element and the actuation element are arranged in series.

25. The actuation assembly of any of examples 19-23 wherein the elastic element and the actuation element are arranged in parallel.

26. The actuation assembly of any of examples 19-23 wherein the actuation element is disposed within the elastic element.

27. The actuation assembly of any of examples 19-26 wherein the actuation element is composed of nitinol.

28. The actuation assembly of any of examples 19-27 wherein the first material state is a martensitic material state, and wherein the second material state is an austenitic material state.

29. A system for shunting blood between a left atrium and a right atrium of a patient, the system comprising:
a shunting element having a lumen extending therethrough, wherein the lumen is configured to fluidly couple the left atrium and the right atrium when the shunting element is implanted in the patient;
a membrane operably coupled to the shunting element and including an aperture at least generally aligned with the lumen; and
an actuation assembly configured to adjust a size of the aperture so as to selectively control blood flow through the lumen, the actuation assembly having an elastic element and a shape memory element operably coupled to the elastic element.

30. The system of example 29 wherein:
the shape memory element has a first spring constant when at a first temperature;
the shape memory element has a second spring constant when at a second temperature above the first temperature, the second spring constant being greater than the first spring constant; and
the elastic element has a third spring constant when at the first temperature, the third spring constant being greater than the first spring constant and less than the second spring constant.

31. The system of example 30 wherein the first temperature is a body temperature of the patient and the second temperature is an elevated temperature resulting from heating of the shape memory element.

32. The system of any of examples 29-31 wherein the shape memory element is configured to transition from a first configuration to a second configuration in response to applied heat to adjust the size of the aperture.

33. The system of example 32 wherein the elastic element is configured to apply a force to the shape memory element that at least partially counteracts transitioning of the shape memory element from the second configuration to the first configuration after the heat has been applied.

34. The system of example 33 wherein the actuation assembly further comprises a locking structure configured to engage one or more of the shape memory element or the elastic element to maintain the shape memory element in the second configuration.

35. The system of example 34 wherein the locking structure comprises one or more ratchets, racks, pins, or teeth.

36. The system of any of examples 32-35, further comprising a ratchet mechanism operably coupled to the actuation assembly, wherein the ratchet mechanism enables the size of the aperture to decrease as the shape memory element transitions from the first configuration to the second configuration while preventing the size of the aperture from increasing as the shape memory element transitions from the second configuration to the first configuration.

37. The system of any of examples 32-35, further comprising a ratchet mechanism operably coupled to the actuation assembly, wherein the ratchet mechanism enables the size of the aperture to increase as the shape memory element transitions from the first configuration to the second configuration while preventing the size of the aperture from decreasing as the shape memory element transitions from the second configuration to the first configuration.

38. The system of any of examples 29-37 wherein the elastic element is connected to the shape memory element in series.

39. The system of any of examples 29-37 wherein the elastic element at least partially surrounds the shape memory element.

### CONCLUSION

Embodiments of the present disclosure may include some or all of the following components: a battery, supercapacitor, or other suitable power source; a microcontroller, FPGA, ASIC, or other programmable component or system capable of storing and executing software and/or firmware that drives operation of an implant; memory such as RAM or ROM to store data and/or software/firmware associated with an implant and/or its operation; wireless communication hardware such as an antenna system configured to transmit via Bluetooth, WiFi, or other protocols known in the art; energy harvesting means, for example a coil or antenna which is capable of receiving and/or reading an externally-provided signal which may be used to power the device, charge a battery, initiate a reading from a sensor, or for other purposes. Embodiments may also include one or more sensors, such as pressure sensors, impedance sensors, accelerometers, force/strain sensors, temperature sensors, flow sensors, optical sensors, cameras, microphones or other acoustic sensors, ultrasonic sensors, ECG or other cardiac rhythm sensors, SpO2 and other sensors adapted to measure tissue and/or blood gas levels, blood volume sensors, and other sensors known to those who are skilled in the art. Embodiments may include portions that are radiopaque and/or ultrasonically reflective to facilitate image-guided implantation or image guided procedures using techniques such as fluoroscopy, ultrasonography, or other imaging methods. Embodiments of the system may include specialized delivery catheters/systems that are adapted to deliver an implant and/or carry out a procedure. Systems may include components such as guidewires, sheaths, dilators, and multiple delivery catheters. Components may be exchanged via over-the-wire, rapid exchange, combination, or other approaches.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments. For example, although this disclosure has been written to describe devices that are generally described as being used to create a path of fluid communication between the LA and RA, the LV and the right ventricle (RV), or the LA and the coronary sinus, it should be appreciated that similar embodiments could be utilized for shunts between other chambers of heart or for shunts in other regions of the body.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A system for shunting fluid between a first body region and a second body region of a patient, the system comprising:
a shunting element having a lumen extending therethrough and configured to fluidly connect the first body region and the second body region when implanted in the patient; and
a flow control element moveable through a plurality of discrete geometries, wherein each discrete geometry is associated with a relative drainage resistance through the lumen, and wherein the flow control element is selectively moveable between the plurality of discrete geometries.

2. The device of claim 1, further comprising an actuation assembly configured to selectively move the flow control element through the plurality of discrete geometries, wherein the actuation assembly includes at least one actuation element and a ratchet mechanism.

3. The device of claim 2 wherein the actuation element and the ratchet mechanism are configured to provide a lock step adjustment to the flow control element to move the flow control element through the plurality of discrete geometries.

4. The device of claim 2 wherein the actuation assembly further includes an engagement member operably coupled to the flow control element and the actuation element, and wherein the engagement member is configured to engage the ratchet mechanism.

5. The device of claim 4 wherein the ratchet mechanism includes a plurality of teeth defining a plurality of grooves therebetween, and wherein the engagement member engages the ratchet mechanism in one or more of the grooves.

6. The device of claim 5 wherein the actuation element is actuatable between a neutral configuration and an actuated configuration, and wherein, when actuated between the neutral configuration and the actuated configuration, (i) the flow control element moves from a first geometry to a second geometry, and (ii) the engagement member moves from a first groove to a second groove.

7. The device of claim 6 wherein, when the actuation element moves from the actuated configuration to the neutral configuration, the flow control element retains the second geometry and the engagement member remains in the second groove.

8. The device of claim 2 wherein the ratchet mechanism has a sawtooth configuration.

9. The device of claim 2 wherein the ratchet mechanism is a one-way ratchet mechanism that is configured to provide the discrete adjustments to the flow control element geometry in a first direction but prevent adjustment to the flow control element in a second direction opposite the first direction.

10. The device of claim 9 wherein the geometry is a diameter, and wherein the discrete adjustments to the flow control element geometry in a first direction comprises making the diameter smaller.

11. The device of claim 9 wherein the ratchet mechanism includes a ramp structure configure to reset the actuation assembly.

12. The device of claim 2 wherein the actuation element comprises a shape memory material.

13. A system for shunting fluid between a first body region and a second body region of a patient, the system comprising:
a shunting element having a lumen extending therethrough and configured to fluidly connect the first body region and the second body region when implanted within the patient, wherein the shunting element includes an adjustable aperture for controlling flow of fluid through the lumen; and
a flow control element moveable through a plurality of discrete positions, wherein each discrete position is associated with a different aperture geometry, and wherein the flow control element is selectively moveable between the plurality of discrete positions.

14. The system of claim 13 wherein the aperture geometry is an aperture diameter.

15. The system of claim 13, further comprising a ratchet mechanism that controls the movement of the flow control element through the plurality of discrete positions.

16. The system of claim 15 wherein the ratchet mechanism is configured to selectively decrease the diameter of the aperture while preventing an increase in the diameter of the aperture.

17. The system of claim 16 wherein the aperture is moveable between a plurality of diameters, with each corresponding diameter smaller than the previous.

18. A device for treating heart failure, the device comprising:
a lumen configured to fluidly connect a left atrium and a right atrium of a heart of a subject;
a flow control element operably coupled to the lumen; and
an actuation assembly configured to alter the flow of fluid through the lumen by adjusting a geometry of the flow control element, wherein the actuation assembly includes—
an actuation element,
a ratchet mechanism having a plurality of grooves, and
an engagement member operably coupled to the actuation element and the flow control element, wherein the engagement member is configured to engage the ratchet mechanism in one or more of the grooves,
wherein actuation of the actuation element causes (i) the flow control element to move from a first geometry to a second geometry, thereby adjusting the flow of fluid through the lumen, and (ii) the engagement member to move from a first groove to a second groove, thereby maintaining the flow control element in the second geometry.

19-39. (canceled)
